(19) United States
(12) Patent Application Publication
(10) Pub. No.: US 2025/0271390 A1
(43) Pub. Date: Aug. 28, 2025

(54) Method and Apparatus for Sensing Free Chlorine and Monochloramine (NH2Cl)

(71) Applicant: Halogen Systems, Inc., Reno, NV (US)

(72) Inventors: Michael A. Silveri, Incline Village, NV (US); Kegan Rahe, Reno, NV (US)

(21) Appl. No.: 19/053,739

(22) Filed: Feb. 14, 2025

Related U.S. Application Data
(60) Provisional application No. 63/556,978, filed on Feb. 23, 2024.

(51) Int. Cl. G01N 27/333; G01N 27/49; G01N 33/18

(52) U.S. Cl. CPC G01N 27/333; G01N 27/49; G01N 33/182

(57) ABSTRACT

A method and an apparatus determine a concentration of monochloramine in a water system. The apparatus is a three-electrode amperometric sensor that receives voltages from a waveform generator. The sensor senses currents responsive to the voltages. The currents are further responsive to concentrations of free chlorine and monochloramine. Current values responsive to the sensed current are provided to a system controller. The waveform generator generates an activation waveform, followed by a free chlorine measurement waveform, followed by a second activation waveform, followed by a combined chlorine measurement waveform. The system controller converts an average of current values obtained when the free chlorine measurement waveform is applied to a free chlorine concentration; converts an average of current values obtained when the combined chlorine measurement waveform is applied to a combined chlorine concentration; and calculates a difference of the two averages as a monochloramine concentration.

### RELATED APPLICATION

This application claims the benefit of priority under 35 USC 119(e) to U.S. Provisional Application No. 63/556,978, filed Feb. 23, 2024, entitled “Method and Apparatus for Sensing Free Chlorine and Monochloramine (NH₂Cl),” which is hereby incorporated by reference in its entirety.

### BACKGROUND

Amperometric sensors have been used for more than forty years for measurement of chlorine concentration in drinking water and wastewater. A few variations have occurred over the years, but the basic technology has not changed much. When chlorine is added to water in any form, the following reaction in Equation (1) occurs:

Cl₂+H₂O=HOCl+H⁺+Cl⁻ (1)

The reaction at the working electrode (cathode) in an amperometric sensor is a reduction of the chlorine to chloride in accordance with the following Equation (2):

HOCl+H⁺+2e⁻→Cl⁻+H₂O (2)

When monochloramine is the desired disinfectant, anhydrous ammonia (NH3 or some compound containing same) is added:

NH₃+HOCl=NH₂Cl+H₂O (3)

The liberated electrons caused by the reduction are measured (in nanoamperes) and are directly proportional to the concentration of chlorine in solution.

Known methods for measuring free chlorine in the presence of monochloramine or excess ammonia are not sufficiently accurate. Monochloramine (NH₂Cl) is listed as an interferent in free chlorine measurements. The presence of monochloramine is a vexing problem for water treatment utilities because free chlorine and monochloramine are quantified to control the chlorination process and the chloramination process. Chlorination using chloramine is gaining popularity with drinking water utilities to minimize chlorination byproducts formed when organic substances like humic acid are present in source water. Oxidation at breakpoint is typically used to oxidize nitrogenous compounds present. Under these conditions, chlorination byproducts like trihalomethanes (THMs) and the five common haloacetic acids (HAA5)—monochloroacetic acid, dichloroacetic acid (DCA), trichloroacetic acid (TCA), monobromoacetic acid, and dibromoacetic acid—can form. Some studies suggest that long term exposure to these compounds may cause undesirable health effects.

Many amperometric sensor manufacturers list monochloramine as an interferent that affects the free chlorine sensor readings of the amperometric sensors. See, for example, CLF10 sc & CLT10 sc Free & Total Reagentless Chlorine Analyzers, LIT2679 Rev S, Hach Company, 2018, which lists monochloramine as an interferent. These residuals even interfere in colorimetric or spectrophotometric DPD free chlorine measurements and are known as the Pink Phantom, as described, for example by Robert Spon in Do You Really Have a Free Chlorine Residual?, Opflow, June 2008, pages 24-27. The DPD spectrophotometric method is widely used globally as is corroborated in several other publications: Monochloramine interference is discussed by Daniel L. Harp on page 15 of Current Technology of Chlorine Analysis for Water and Wastewater, Technical Information Series—Booklet No. 17, Hach Company, 2002: “There is considerable controversy about monochloramine interference in the free chlorine DPD test. Some studies (Ref.3.14) have indicated the percent interference in the free chlorine results can vary from 2.6 to 6.0%, depending on the monochloramine concentration and sample temperature.” The inadequacy of known methods is discussed by Vadim B. Malkov and Gary B. Visser on page 25 of Discovering the Unexpected in Disinfection Applications, Opflow, May 2011, pages 24-32: “Currently, no ideal method to quantify chlorine and chloramines in water exists. All common methods of chlorine analysis display some lack of specificity and aren't selective enough to be interference free.” See also page 4 of Vadim B. Malkov and Gary B. Visser in Uncovering the Unexpected In Drinking Water Disinfection Applications Using Continuous Monitoring, Application Note: Amperometric vs. Colorimetric Comparison, Hach Company, 2020.

The interferences caused by chloramines seem to occur near the zero point. In recent tests, the tested methods did not go to zero free chlorine even in the presence of excess added ammonia, which drives the measured species to monochloramine.

On page 102 of Improved free chlorine amperometric sensor chip for drinking water applications, Analytica Chemica Acta, 554, pages 98-104, 2005, F. Javier Del Campo et. al disclose the polarization of on-chip gold and platinum disc electrodes at 0.15 volts, which is the potential for two electron mass transport controlled reduction of hypochlorite/hypochlorous acid. The 0.15-volt potential is widely used in amperometric sensors; however, measurements at this potential do not differentiate free chlorine from monochloramine.

U.S. Pat. No. 8,887,556 to Silveri for Amperometric Sensor System discloses a fixed measurement potential of 0.25 volt for 60 seconds followed by stabilization pulse of -0.6 volt for 2 seconds or more. See column 21 and FIG. 28.

U.S. Pat. No. 8,881,581 to Silveri for Amperometric Sensor System discloses a previous attempt to determine the presence of chloramine (NH₂Cl) by examining the ratio of current at two different fixed potentials of 0.1 volt and 0.3 volt. See column 3 at lines 41-50.

Other systems have used adjusting the pH of solution to speciate some fractions of chlorine. See, for example, International Publication No. WO 2009/055093 to Fabrizzio et al. for Electrochemical Methods for Selective Detection of Free Chlorine, Monochloramine and Dichloramine.

Reagent feed systems use colorimeters and chromogenic reagents to determine total chlorine, free ammonia and monochloramine; however, reagent feed systems are expensive to purchase and operate.

Certain systems for measuring monochloramine in biofilms use a membrane to prevent interference with dissolved oxygen. For example, U.S. Pat. No. 9,586,814 to Pressman et al., for Solid State Amperometric Chlorine Sensor uses a measurement potential of +0.15 volt to +0.45 volt.

U.S. Pat. No. 7,189,314 to Pace et al. for Method and Apparatus for Quantitative Analysis discloses a sensor chip with membranes for various measurements.

U.S. Pat. No. 7,087,150 to Feng for Chloramine Amperometric Sensor uses a membrane to measure chloramine using a gas diffusion membrane method and a porous working electrode.

US Patent Application Publication No. 2013/0313128 to Fielden et al. for Electrochemical Sensor Apparatus and Electrochemical Sensing Method discloses a boron-doped diamond (BDD) sensor using anodic oxidation and cathode reduction to speciate chlorine.

In Differentiation and Quantification of Free Chlorine and Inorganic Chloramines in Aqueous Solution by MIMS, Chii Shang and Ernest R. Blatchley III disclose a membrane introduction mass spectrometric (MIMS) method for differentiation and quantification of free chlorine and inorganic chloramines in aqueous solution using membrane columns in a low-cost mass spectrometer.

U.S. Pat. No. 11,084,738 to Blatchley III for Methods and Systems for Separation of Chloramines in Aqueous Solutions discloses a test kit design using chemical reagents and a permeable membrane for separation of chloramine species.

The chemistry of chloramines is fairly well understood and can be summarized in its simplest form by three reversible reactions involving ammonia (NH3), hypochlorous acid (HOCl), monochloramine (NH₂Cl), dichloramine (NHCl₂) and trichloramine (NHCl₃):

NH₃+HOCl [symbol] NH₂Cl+H₂O (4)

NH₂Cl+HOCl [symbol] NHCl₂+H₂O (5)

NHCl₂+HOCl [symbol] NHCl₃+H₂O (6)

Monochloramine is generally the dominant species produced during drinking-water disinfection. The production of dichloramine is favored as the chlorine-to-nitrogen ratio increases and the pH decreases. Hydrolysis reactions (the reverse reactions in Equations (4), (5) and (6) above) are also of considerable interest because they generate hypochlorous acid, which may be important in the formation of the chlorine-substituted organic compounds hypobromous acid-hypobromite and bromamines. Also, the hydrolysis reaction with monochloramine liberates ammonia. At equilibrium, no more than several percent of monochloramine is hydrolysed to free chlorine. See, Alicia C. Diehl et al., “DBP formation during chloramination,” Journal AWWA, 1 Jun. 2000.

In addition to hydrolysis and conversion to trichloramine (Equation (6) above), dichloramine decomposes by several other reactions, two of which are base-catalysed. The decomposition of dichloramine accelerates as pH increases, the concentration of bases (e.g., alkalinity) increases, or both increase. Therefore, dichloramine is much less stable than monochloramine under most conditions of practical interest; however, the greater instability of dichloramine does not necessarily mean that dichloramine is of little significance in the formation of disinfection by-products. See, Diehl et al., cited above.

Monochloramine decomposes in a stepwise fashion. The monochloramine is converted first to dichloramine. The subsequent decomposition of dichloramine is primarily responsible for loss of total residual chlorine. The two major pathways for the decomposition of monochloramine are hydrolysis of monochloramine and subsequent reaction with free chlorine (Equations (5) and (6) above) and general acid catalysis in accordance with the following reversible reaction:

NH₂Cl+NH₂Cl+H⁺ [symbol] NHCl₂+NH₃+H⁺ (7)

The rate of general acid catalysis (Equation (7)) is a function of the concentration of proton donors. The rate increases as the concentration increases and as pH decreases. The carbonate system, via carbonic acid and bicarbonate, may significantly increase the rate of acid-catalysed decomposition at concentrations and pH values typical of many drinking-water systems. Therefore, the decomposition rate of both monochloramine and dichloramine may increase as alkalinity increases. See, Diehl et al., cited above.

Data on the production of monochloramine for water treatment are not available.

A need exists for an accurate method for measuring free chlorine in the presence of monochloramine or excess ammonia.

### SUMMARY

An object of the method disclosed herein is to improve the measurement of monochloramine in the presence of free chlorine. One aspect of the disclosure herein is the measurement of free chlorine without a monochloramine interference. Another object of the method disclosed herein is to measure both monochloramine and free chlorine on the same sensor by changing the measurement potential. The measurements are performed without using reagents and without using membranes. The measurements only use bare electrodes. The method is simple, reliable, easy to use, and robust. In certain embodiments, the method is implemented on an amperometric sensor system such as the system disclosed in U.S. Pat. No. 8,887,556 to Silveri for Amperometric Sensor System, which is incorporated by reference herein. The method can be used either with fixed measurements or pulsed amperometric detection (PAD).

One aspect of the embodiments disclosed herein is a method and an apparatus determine a concentration of monochloramine in a water system. The apparatus is a three-electrode amperometric sensor that receives voltages from a waveform generator. The sensor senses currents responsive to the voltages. The currents are further responsive to concentrations of free chlorine and monochloramine. Current values responsive to the sensed current are provided to a system controller. The waveform generator generates an activation waveform, followed by a free chlorine measurement waveform, followed by a second activation waveform, followed by a combined chlorine measurement waveform. The system controller converts an average of current values obtained when the free chlorine measurement waveform is applied to a free chlorine concentration; converts an average of current values obtained when the combined chlorine measurement waveform is applied to a combined chlorine concentration; and calculates a difference of the two averages as a monochloramine concentration.

Another aspect in accordance with the embodiments disclosed herein is a method of measuring free chlorine (HOCl) without substantial interference of monochloramine (NH₂Cl).

Another aspect in accordance with the embodiments disclosed herein is a method for determining a concentration of free chlorine in water using a three-electrode amperometric sensor with a bare working electrode. The amperometric sensor includes a potentiostat with selectable voltages. The method comprises executing an activation procedure. The activation procedure comprises applying an activation voltage sequence to the working electrode. The activation voltage sequence comprises a plurality of activation cycles, wherein each activation cycle comprises a first activation voltage followed by a second activation voltage. The method further comprises applying a free chlorine measurement sequence to the working electrode. The free chlorine measurement sequence comprises a plurality of free chlorine measurement cycles, wherein each free chlorine measurement cycle comprises a first free chlorine measurement voltage followed by a second free chlorine measurement voltage followed by a third free chlorine measurement voltage. While the first free chlorine measurement voltage is applied in each free chlorine measurement cycle, the method comprises measuring a sensed current and saving a respective free chlorine measurement value responsive to the sensed current for each cycle. The method averages the saved free chlorine measurement values to obtain an average free chlorine measurement value; and the method converts the average free chlorine measurement value to a value representing a concentration of free chlorine.

Another aspect in accordance with the embodiments disclosed herein is a method for determining a concentration of monochloramine in water using a three-electrode amperometric sensor with a bare working electrode. The amperometric sensor includes a potentiostat with selectable voltages. The method comprises executing an activation procedure. The activation procedure comprises applying an activation voltage sequence to the working electrode. The activation voltage sequence comprises a plurality of activation cycles, wherein each activation cycle comprises a first activation voltage followed by a second activation voltage. The method further comprises measuring free chlorine concentration by applying a free chlorine measurement sequence to the working electrode. The free chlorine measurement sequence comprises a plurality of free chlorine measurement cycles, wherein each free chlorine measurement cycle comprises a first free chlorine measurement voltage followed by a second free chlorine measurement voltage followed by a third free chlorine measurement voltage. While the first free chlorine measurement voltage is applied in each free chlorine measurement cycle, the method comprises measuring a sensed current and saving a respective free chlorine measurement value responsive to the sensed current for each cycle. The method averages the saved free chlorine measurement values to obtain an average free chlorine measurement value; and the method converts the average free chlorine measurement value to a value representing a concentration of free chlorine. The method repeats the activation procedure. The method further comprises measuring combined chlorine concentration by applying a combined chlorine measurement sequence to the working electrode. The combined chlorine measurement sequence comprises a plurality of combined chlorine measurement cycles, wherein each combined chlorine measurement cycle comprises a first combined chlorine measurement voltage followed by a second combined chlorine measurement voltage followed by a third combined chlorine measurement voltage. While the first combined chlorine measurement voltage is applied in each combined chlorine measurement cycle, the method comprises measuring a sensed current and saving a respective combined chlorine measurement value responsive to the sensed current for each cycle. The method averages the saved combined chlorine measurement values to obtain an average combined chlorine measurement value; and the method converts the average combined chlorine measurement value to a value representing a concentration of combined chlorine. The method subtracts the value representing the concentration of free chlorine from the value representing the concentration of combined chlorine to determine a concentration of monochloramine.

Another aspect in accordance with the embodiments disclosed herein is a method for determining a concentration of monochloramine in water using a three-electrode amperometric sensor with a bare working electrode, the amperometric sensor including a potentiostat with selectable voltages. The method comprises executing an activation procedure wherein the activation procedure comprises applying a first activation voltage to the working electrode for a first activation step time duration; applying a second activation voltage to the working electrode for a second activation step time duration; and repeatedly applying the first activation voltage and the second activation voltage for a first plurality of times. The method further comprises measuring free chlorine concentration. Measuring free chlorine concentration comprises applying a first free chlorine measurement voltage to the working electrode for a first free chlorine measurement duration. While the first free chlorine measurement voltage is applied, the method measures a sensed current and saves a value responsive to the sensed current. The method further comprises applying a second free chlorine measurement voltage to the working electrode for a second free chlorine measurement duration; applying a third free chlorine measurement voltage to the working electrode for a third free chlorine measurement duration. The method repeatedly applies the first chlorine measurement voltage, measures the sensed current, applies the second chlorine measurement voltage, and applies the third chlorine measurement voltage for a second plurality of times. The method averages the saved values responsive to the sensed current to obtain a first average sensed current value; and converts the first average sensed current value to a value representing a concentration of free chlorine. The method repeats the activation procedure. The method further comprises measuring combined chlorine concentration. Measuring combined chlorine concentration comprises applying a first combined chlorine measurement voltage to the working electrode for a first combined chlorine measurement duration. While the first combined chlorine measurement voltage is applied, the method measures a sensed current and saves a value responsive to the sensed current. The method further comprises applying a second combined chlorine measurement voltage to the working electrode for a second combined chlorine measurement duration; applying a third combined chlorine measurement voltage to the working electrode for a third combined chlorine measurement duration. The method repeatedly applies the first combined chlorine measurement voltage, measures the sensed current, applies the second chlorine measurement voltage, and applies the third chlorine measurement voltage for a second plurality of times. The method averages the saved values responsive to the sensed current to obtain a second average sensed current value; and converts the second average sensed current value to a value representing a combined concentration of free chlorine and monochloramine. The method subtracts the value representing the concentration of free chlorine from the value representing the combined concentration of free chlorine and monochloramine to determine a concentration of monochloramine.

In certain embodiments in accordance with this aspect, the first activation voltage is approximately −1.5 volts; and the second activation voltage is approximately 0.0 volt. In certain embodiments, the first activation step time duration is approximately 100 milliseconds; and the second activation step time duration is approximately 100 milliseconds. In certain embodiments, the first plurality of times is 10.

In certain embodiments in accordance with this aspect, the first free chlorine measurement voltage is in a range of approximately 0.35 volt to approximately 0.45 volt; the second free chlorine measurement voltage is approximately 0.8 volt; and the third free chlorine measurement voltage is approximately −1.15 volts. In certain embodiments, the first free chlorine measurement duration is approximately 430 milliseconds; the second free chlorine measurement duration is approximately 200 milliseconds; and the third free chlorine measurement duration is approximately 230 milliseconds. In certain embodiments, the second plurality of times is 15-30.

In certain embodiments in accordance with this aspect, the first combined chlorine measurement voltage is in a range of approximately −0.15 volt to approximately 0.0 volt; the second combined chlorine measurement voltage is approximately 0.8 volt; and the third combined chlorine measurement voltage is approximately −1.15 volts. In certain embodiments, the first combined chlorine measurement duration is approximately 430 milliseconds; the second combined chlorine measurement duration is approximately 200 milliseconds; and the third combined chlorine measurement duration is approximately 230 milliseconds. In certain embodiments, the third plurality of times is 15-30.

Another aspect in accordance with the embodiments disclosed herein is a method for determining a concentration of monochloramine in water using a three-electrode amperometric sensor with a bare working electrode. The amperometric sensor includes a potentiostat with selectable voltages. The method comprises executing an activation procedure wherein the activation procedure comprises 10 activation cycles. Each activation cycle comprises applying −1.5 volts to the working electrode for 100 milliseconds; and applying 0.0 volt to the working electrode for 100 milliseconds. The method further comprises executing a free chlorine concentration measurement procedure, which comprises executing 15-30 free chlorine measurement cycles. Each free chlorine measurement cycle comprises applying a voltage in a range of approximately 0.35 volt to approximately 0.45 volt to the working electrode for 430 milliseconds as a free chlorine measurement voltage. While the free chlorine measurement voltage is applied, the method measures a sensed current and saves a value responsive to the sensed current. The free chlorine measurement cycle further comprises applying 0.8 volt to the working electrode for 200 milliseconds; and applying −1.15 volts to the working electrode for 230 milliseconds. The free chlorine concentration measurement procedure further comprises averaging the saved values responsive to the sensed current to obtain a first average sensed current value; and converting the first average sensed current value to a value representing a concentration of free chlorine. The method repeats the activation procedure. The method further comprises executing a combined chlorine concentration measurement procedure, which comprises executing 15-30 combined chlorine measurement cycles. Each combined chlorine measurement cycle comprises applying a voltage in a range of approximately −0.15 volt to approximately 0.0 volt to the working electrode for 430 milliseconds as a combined chlorine measurement voltage. While the combined chlorine measurement voltage is applied, the method measures a sensed current and saves a value responsive to the sensed current. The combined chlorine concentration measurement cycle further comprises applying 0.8 volt to the working electrode for 200 milliseconds; and applying −1.15 volts to the working electrode for 230 milliseconds. The combined chlorine concentration measurement procedure further comprises averaging the saved values responsive to the sensed current to obtain a second average sensed current value; and converting the second average sensed current value to a value representing a concentration of combined chlorine. The method further comprises subtracting the value representing the concentration of free chlorine from the value representing the concentration of combined chlorine to determine a concentration of monochloramine.

Another aspect in accordance with the embodiments disclosed herein is an amperometric system for determining concentrations of free chlorine and monochloramine in water. The system comprises a three-electrode potentiostat, which comprises a measurement cell having a working electrode, a reference electrode and a counter electrode, the electrodes insertable into a source of water; circuitry responsive to an input voltage to generate a current through the measurement cell to cause a voltage between the working electrode and the reference electrode responsive to the input voltage; and a current sensor that senses the current through the measurement cell. The system also includes a waveform generator having an output coupled to the potentiostat. The waveform generator generates the input voltage as a first waveform, a second waveform, a third waveform, and a fourth waveform applied in sequence. The first waveform comprises 10 cycles wherein each cycle comprises a first half cycle at −1.5 volts for 100 milliseconds and a second half cycle of 0.0 volt at 100 milliseconds. The second waveform comprises 15-30 cycles wherein each cycle comprises a first cycle portion, a second cycle portion and a third cycle portion. The first cycle portion comprises a voltage in a range of approximately 0.35 volt to approximately 0.45 volt applied for 430 milliseconds. The second cycle portion comprises 0.8 volt applied for 200 milliseconds. The third cycle portion comprises −1.15 volts applied for 230 milliseconds. The third waveform comprises 10 cycles wherein each cycle comprises a first half cycle at −1.5 volts for 100 milliseconds and a second half cycle of 0.0 volt at 100 milliseconds. The fourth waveform comprises 15-30 cycles wherein each cycle comprises a first cycle portion, a second cycle portion and a third cycle portion,. The first cycle portion comprises a voltage in a range of approximately −0.15 volt to approximately 0.0 volt applied for 430 milliseconds. The second cycle portion comprises 0.8 volt applied for 200 milliseconds. The third cycle portion comprises −1.15 volts applied for 230 milliseconds. The system further includes a system controller coupled to the potentiostat to receive values from the current sensor. The system controller is configured to receive and save a respective value responsive to a concentration of free chlorine during each first cycle portion of the second waveform. The system controller averages the saved values to determine a first average sensed current value. The system controller converts the first average sensed current value to a value representing the concentration of free chlorine. The system controller is further configured to receive and save a respective value responsive to a concentration of total chlorine during each first cycle portion of the fourth waveform. The system controller averages the saved values to determine a second average sensed current value. The system controller converts the second average sensed current value to a value representing a concentration of combined chlorine and monochloramine. The system controller subtracts the value representing the concentration of free chlorine from the value representing the concentration of combined chlorine and monochloramine to determine a concentration of monochloramine.

Another aspect in accordance with the embodiments disclosed herein is a method for determining a first concentration of a first parameter and determining a second concentration of a second parameter in water using a three-electrode amperometric sensor with a bare working electrode. The amperometric sensor includes a potentiostat with selectable voltages. The method comprises executing an activation procedure comprising applying an activation voltage sequence to the working electrode. The activation voltage sequence comprises a plurality of activation cycles. Each activation cycle comprises a first activation voltage followed by a second activation voltage. The method further comprises measuring the first concentration of the first parameter by applying a first parameter measurement sequence to the working electrode. The first parameter measurement sequence comprises a plurality of first parameter measurement cycles. Each first parameter measurement cycle comprises an initial first parameter measurement voltage followed by a second first parameter measurement voltage followed by a third first parameter measurement voltage. While the initial first parameter measurement voltage is applied in each first parameter measurement cycle, the method measures a sensed current and saves a respective first parameter measurement value responsive to the sensed current for each cycle. The method averages the first parameter measurement values to obtain an average first parameter measurement value. The method converts the average first parameter measurement value to a value representing a first concentration of the first parameter. The method repeats the activation procedure. The method further measures a combined concentration of the first parameter and the second parameter by applying a combined concentration measurement sequence to the working electrode. The combined concentration measurement sequence comprises a plurality of combined concentration measurement cycles. Each combined concentration measurement cycle comprises a first combined concentration measurement voltage followed by a second combined concentration measurement voltage followed by a third combined concentration measurement voltage. While the first combined chlorine measurement voltage is applied in each combined concentration measurement cycle, the method measures a sensed current and saves a respective combined concentration measurement value responsive to the sensed current for each cycle. The method averages the saved combined concentration measurement values to obtain an average combined concentration measurement value. The method converts the average combined concentration measurement value to a value representing a combined concentration of the first parameter and the second parameter. The method subtracts the value representing the concentration of the first parameter from the value representing the combined concentration of the first parameter and the second parameter to determine a concentration of the second parameter.

In certain embodiments in accordance with this aspect, the first parameter is free chlorine and the second parameter is monochloramine.

### BRIEF DESCRIPTIONS OF THE DRAWINGS

FIG. 1 illustrates a perspective view of an embodiment of a sensor system as viewed from the proximal end of the sensor system.

FIG. 2 illustrates a perspective view of an embodiment of the sensor system of FIG. 1 as viewed from the distal end of the sensor system.

FIG. 3 illustrates a block diagram of the electronics system of the sensor system of FIGS. 1 and 2.

FIG. 4 illustrates a typical potentiostat configuration using buffers to isolate a driving amplifier and a reference electrode.

FIG. 5 illustrates a potentiostat configuration having a waveform generator that provides a sequence of voltage potentials to the input amplifier of the potentiostat of FIG. 4.

FIG. 6 illustrates a waveform generated by the waveform generator of FIG. 5 comprising an activation sequence followed by a pulsed amperometric detection (PAD) sequence, the PAD sequence comprising multiple voltage cycles wherein each voltage cycle has three voltage levels selected to enable measurement of free chlorine concentration.

FIG. 7 illustrates one cycle of the activation sequence of FIG. 6.

FIG. 8 illustrates one cycle of the PAD sequence of FIG. 6.

FIG. 9 illustrates a waveform generated by the waveform generator of FIG. 5 comprising an activation sequence followed by a PAD sequence, the PAD sequence comprising multiple voltage cycles wherein each voltage cycle has three voltage levels selected to enable measurement of combined chlorine concentration.

FIG. 10 illustrates one cycle of the PAD sequence of FIG. 9.

FIG. 11 illustrates the waveform of FIG. 6 and the waveform of FIG. 9 generated in sequence to enable measurement of the combined chlorine concentration immediately after the measurement of the free chlorine concentration.

FIG. 12 illustrates a flow chart of the process for measuring free chlorine concentration and combined chlorine concentration to determine a monochloramine concentration.

FIG. 13 illustrates a flow chart of the activation sequence of FIG. 12.

FIG. 14 illustrates a flow chart of the free chlorine measurement sequence of FIG. 12.

FIG. 15 illustrates a flow chart of the combined chlorine measurement sequence of FIG. 12.

FIG. 16 illustrates graphs of measurements of free chlorine and combined chlorine using the apparatus and method disclosed herein and also illustrates graphs of measurements of free chlorine and monochloramine using a commercially available chlorimination tester.

### DETAILED DESCRIPTION

The method disclosed herein enables accurate measurement of free chlorine in the presence of monochloramine or excess ammonia.

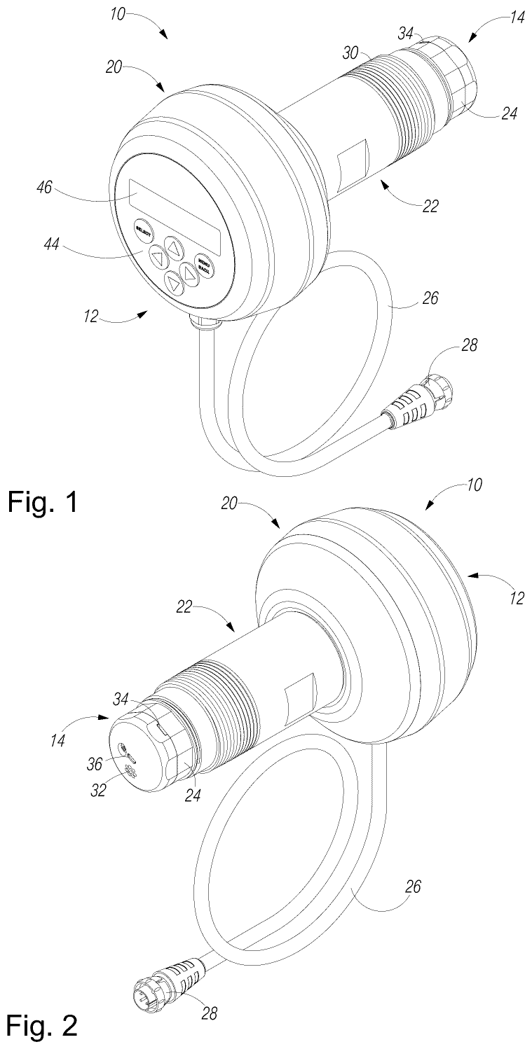

FIG. 1 illustrates a perspective view of an embodiment of a sensor system 10 that can be used to implement the method disclosed herein. The sensor system is disclosed in applicant's prior U.S. Pat. No. 8,887,556 for “Amperometric Sensor System,” which is incorporated herein by reference. The following description illustrates certain aspects of the sensor system disclosed in the prior patent. More detailed information is presented in the patent.

The sensor system 10 extends from a proximal end 12 to a sensor end 14. The sensor system comprises an electronics enclosure 20 at the proximal end, an intermediate housing 22, a sensor housing 24 at the sensor end, and a communications cable 26. The communications cable is terminated at a free end with a connector 28. In the illustrated embodiment, the connector is a conventional M12 connector. The opposite end of the communications cable enters the electronics enclosure. Wires within the communications cable are terminated on a printed circuit board (not shown) within the electronics enclosure, which supports the electronic circuitry described below.

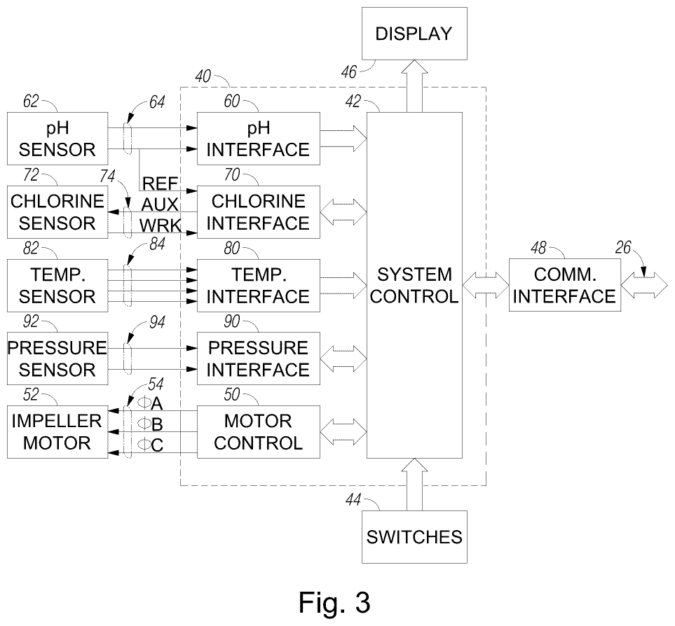

FIG. 2 illustrates a perspective view of the sensor system 10 of FIG. 1 viewed from the sensor end 14. The sensor end of the sensor system includes the sensor housing 24, which extends from the intermediate housing 22. The sensor housing encloses a plurality of sensor elements (not shown). The sensor housing also encloses a flow generator, which includes a motor and an impeller (not shown). The sensor elements and the flow generator are described in detail in applicant's U.S. Pat. No. 8,887,556 and are shown schematically in FIG. 3, which is described below. A threaded portion 30 of the intermediate housing includes threads. As described in U.S. Pat. No. 8,887,556, the threaded portion can be threaded into a pipe fitting or into a container containing water to position the sensor housing within the water. Water enters the sensor housing via a first bore 32 and exits the sensor housing via a first outlet port 34 and a second outlet port 36. Movement of the water into and out of the sensor housing may be increased by selectively activating the motor to drive the impeller.

The sensors and the motor are shown schematically in FIG. 3, which illustrates a simplified block diagram 40 of the electrical circuitry within the electronics enclosure 20 and the sensor housing 24. The electrical circuitry includes a system controller (control processor) 42, which is responsive to a plurality of membrane switches 44 on the electronics enclosure (see FIG. 1) and which displays status and other information on a display 46 on the electronics enclosure (see FIG. 1). The system controller communicates bidirectionally with a monitoring system (not shown) via a communications interface 48, which is coupled to the communications cable 26 (FIGS. 1 and 2). The communications interface may operate with various communications standards. In the embodiment disclosed herein, the communications interface is configured as a conventional RS485 interface. In certain embodiments, the system controller may also communicate via a wireless interface such as a Bluetooth® interface (not shown).

The system controller 42 is coupled to a motor controller 50, which generates three motor phase signals ΦA, ΦB and ΦC. The motor controller provides the motor phase signals to a motor 52 within the sensor housing 24 via a three-wire cable 54 in a selected sequence to control the rotation of the motor and to thereby control the impeller shown in applicant's U.S. Pat. No. 8,887,556.

The system controller 42 is coupled to a pH sensor interface 60 that monitors a pH sensor 62 via a cable 64. For example, the pH sensor interface advantageously includes an analog-to-digital converter to convert the measured voltages from the pH sensor into a digital representation of the measured voltages.

The system controller 42 is coupled to a chlorine sensor interface 70 that performs amperometric measurements. The chlorine sensor interface applies voltages to a chlorine sensor 72 on an auxiliary (AUX) electrode wire in a chlorine sensor cable 74 and receives a measurable current from the chlorine sensor via a working (WRK) electrode wire in the chlorine sensor cable. The chlorine sensor interface shares a reference (REF) electrode wire with the pH sensor interface 60 via the pH sensor cable 64. The operation of the chlorine sensor to apply a voltage to the auxiliary electrode while monitoring the reference voltage and measuring the resulting current on the working electrode is well known to a person skilled in the art.

The system controller 42 is coupled to a temperature interface 80 that monitors the temperature readings on a temperature sensor 82 via a temperature sensor cable 84.

The system controller 42 may also be coupled to a pressure interface 90 that monitors water pressure on a pressure sensor 92 via a pressure sensor cable 94.

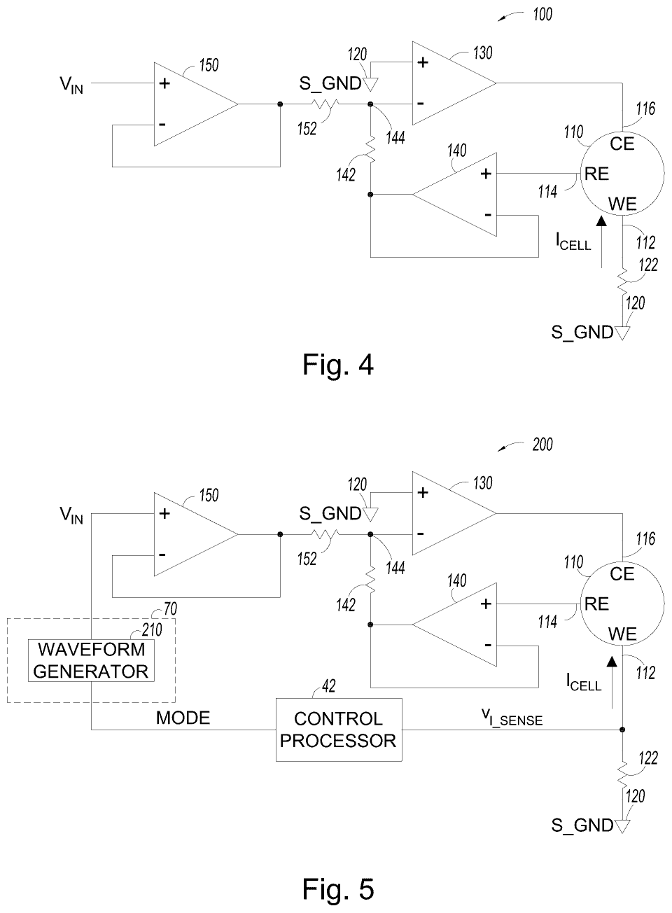

FIG. 4 illustrates an embodiment of a typical potentiostat 100 for measuring chlorine concentration in accordance with the foregoing equations. The potentiostat represents an implementation of the chlorine sensor interface 70 and the chlorine sensor 72 of FIG. 3. The potentiostat includes a measurement cell 110 that includes a working electrode (WE) 112, a reference electrode (RE) 114 and a counter (or auxiliary) electrode (CE) 116. In the illustrated embodiment, the working electrode is a bare electrode comprising gold.

The working electrode 112 is coupled to a signal ground (S_GND) reference 120 via a current sensing resistor 122, which is a resistor having a very low resistance (e.g., 0.1 ohm or less). The signal ground reference may be a conventional circuit ground reference at zero volts or may be an offset voltage reference. For example, in the illustrated potentiostat having circuits connected to a single 5-volt power supply, the signal ground reference may be set at approximately one-half the supply voltage (e.g., approximately 2.5 volts). In other embodiments having components connected between a positive supply voltage and a negative supply voltage, the signal ground reference is also the circuit ground reference. In the following description directed to the illustrated embodiment, a reference to 0 volt is a reference to the signal ground reference. The other signal voltages discussed herein and in the claims are positive (greater than) or negative (less than) with respect to the signal ground reference.

An output of a driving amplifier 130 is coupled to the counter electrode 116. The driving amplifier drives the counter electrode to a voltage with respect to the working electrode to allow a current to flow between the working electrode 112 and the counter electrode. The current flowing through the measurement cell causes a reference voltage to develop on the reference electrode 114 with respect to the working electrode responsive to the magnitude of the current. An output amplifier 140 buffers the reference voltage and provides a buffered reference voltage corresponding to the magnitude of the reference voltage to a summing node 144 via a first summing node resistor 142. An input voltage (V_{IN}) is buffered by an input amplifier 150 to provide a buffered input voltage. The buffered input voltage is provided to the summing node via a second summing node resistor 152. A summed voltage on the summing node voltage is responsive to a difference between the buffered reference voltage and the buffered input voltage.

The summed voltage on the summing node 144 is coupled to an inverting (−) input of the driving amplifier 130. A noninverting (+) input of the driving amplifier is coupled to the signal ground reference 120. The driving amplifier is responsive to the summed voltage on the summing node to vary the output voltage applied to the counter electrode 116 until the measured voltage on the reference electrode 114 is substantially equal to the input voltage to cause the summed voltage on the inverting input to be substantially equal to the voltage of the signal ground reference. The potentiostat operates in a known manner to maintain the applied voltage on the working electrode 112 versus the reference electrode equal to the input voltage V_{IN} by varying the current through the counter electrode. The magnitude of the current (I_{CELL}) flowing through the cell from the working electrode to the counter electrode is measured by sensing a voltage across the voltage sensing resistor 122. The magnitude of the current is determined by the electrons released by reducing hypochlorous acid (HOCl) at the working electrode in accordance with Equation (2). The current is proportional to the reduction and thus proportional to the concentration of chlorine in the water.

FIG. 5 illustrates a modified potentiostat 200 corresponding in part to the potentiostat 100 of FIG. 4. In addition to the elements of the previously described potentiostat, the modified potentiostat includes the chlorine interface 70 of FIG. 3. The chlorine sensor includes a waveform generator 210 that generates waveforms that are applied as the input voltage (V_{IN}) to the input of the input amplifier 150 of the potentiostat. The waveform generator may comprise a microprocessor, a field programmable gate array (FPGA), a custom application specific integrated circuit (ASIC), a plurality of logic circuits or the like that produce the waveforms described below. The waveform generator is responsive to a mode (MODE) signal from the system controller 42 of FIG. 3 to switch between at least a first mode and a second mode. The system controller receives a sensed voltage (V_{I_SENSE}) generated across the sensing resistor 122. The sensed voltage is proportional to the current I_{CELL} through the measurement cell 110. As described below, the waveform generator generates a first waveform in a first mode and generates a second waveform in a second mode. The selected waveform from the waveform generator is applied to the input of the driving amplifier 130 in the potentiostat 200. The potentiostat operates as described above, to maintain the voltage between the working electrode 112 and the reference electrode 114 equal to the applied input voltage V_{IN} generated by the waveform generator. Accordingly, the voltage between the working electrode and the reference electrode varies with the input voltage.

The following description is directed to an improved method of operating the above-described amperometric sensor system in the presence of monochloramine in the water being monitored. The method produces two calibrated values using two separate measurement sequences using pulsed amperometric detection (PAD). Two voltage potentials are used to enable the quantification of free chlorine without interference from monochloramine. At the first voltage potential (e.g., 0.40 volt, which may range from approximately 0.35 volt to approximately 0.45 volt), the response to chloramines is effectively zero such that the measurement indicates only the quantity of free chlorine. At the second voltage potential (e.g., −0.15 volt, which may range from approximately −0.15 volt to approximately 0.0 volt), the measurement includes the responses to both free chlorine and chloramines. The two measurements are used to determine the quantity of chloramines.

The method described below does not require any reagents and may incorporate known methods of cleaning of the electrodes such as the methods described above. The method described below does not require membranes and may be used with bare electrodes. The method described below measures monochloramine in the presence of free chlorine; and measures both monochloramine and free chlorine using the same sensor by changing the measurement potential. The method described below is based in part on reactions that produce free electrons, which are detected by the amperometric sensor.

Free chlorine is reduced according to the following reaction:

HOCl+H⁺+2e⁻=Cl⁻+H₂O (8)

Equation (8) corresponds to Equation (2) discussed above.

Monochloramine is reduced according to the following reaction:

NH₂Cl+2H⁺+2e⁻=NH⁺₄+Cl⁻ (9)

### Detection of Free Chlorine Without Interference From Monochloramine

Water may contain free chlorine only or monochloramine only or both free chlorine and monochloramine. By applying a potential in a range of approximately 0.35 volt to approximately 0.45 volt, the free chlorine residual can be isolated from combined chlorine (primarily monochloramine). In the following description, a potential of 0.40 volt is used. Measurement at this potential assures that only free chlorine is measured. This first step provides speciation of the free chlorine fraction in the presence of monochloramine.

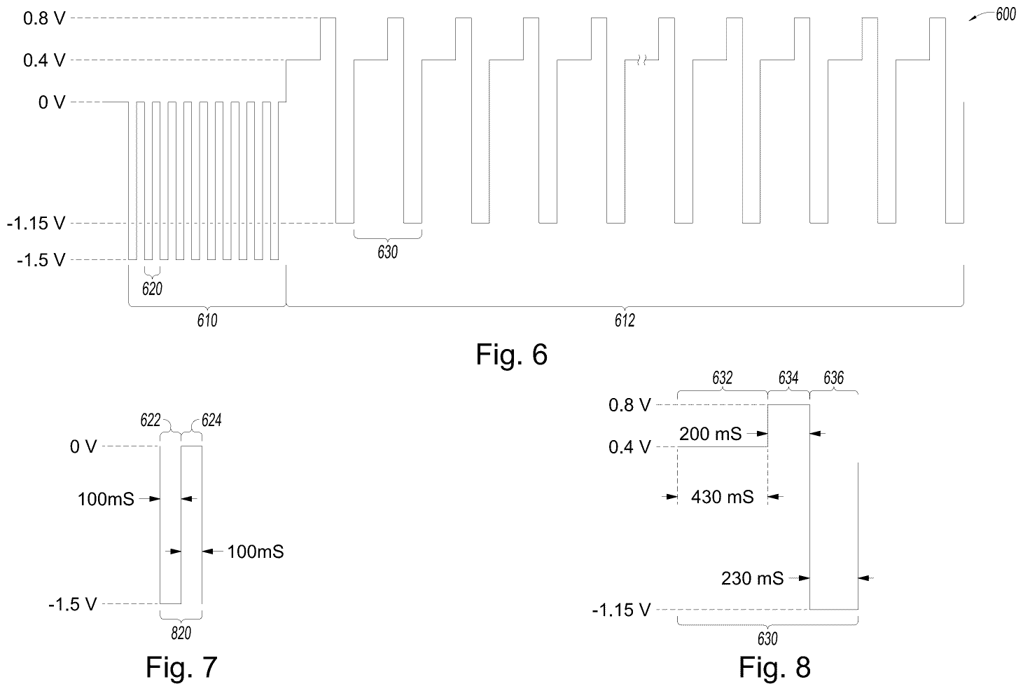

The first step of determining the free chlorine fraction is illustrated by the applied voltages of a waveform 600 in FIG. 6. The waveform is generated by the waveform generator 210 of FIG. 5 in response to commands from the system controller 42 of FIG. 5. The waveform comprises an activation sequence 610 followed by a free chlorine measurement sequence 612.

As illustrated in FIG. 6, the activation sequence comprises 10 cycles 620 wherein each cycle has a period of approximately 200 milliseconds. As shown in FIG. 7 for one cycle, each cycle of the activation sequence comprises two half cycles. A first half cycle 622 of each cycle of the activation sequence comprises an applied voltage of approximately −1.5 volts having a duration of approximately 100 milliseconds. A second half cycle 624 of each cycle of the activation sequence comprise an applied voltage of approximately 0.0 volt having a duration of 100 milliseconds. The number of cycles in the activation sequence may vary with different embodiments; however, a sequence of 10 cycles has been found to be sufficient in the illustrated embodiment. The activation sequence cleans the working electrode through the evolution of hydrogen at −1.5 volts. Coatings (both inorganic and organic) are sloughed off the surface during this step at the selected negative voltage. This method can be used on either platinum or gold working electrodes with a change in the measurement potential.

The free chlorine measurement sequence 612 of FIG. 6 comprises a plurality of cycles 630 wherein each cycle comprises a three portions. The number of cycles may vary with different embodiments. For example, in certain embodiments, the number of cycles in the sequence may vary from 15 to 30. A sequence of 15 cycles has been found to be sufficient in the illustrated embodiment. Although 15 cycles are described in the following description, the number of cycles can be increased to increase the number of measurements in each sequence. Only 10 of the 15 cycles are illustrated in FIG. 6 with a curled break symbol representing the missing 5 cycles. As shown in FIG. 8 for one cycle of the free chlorine measurement sequence, a first portion 632 of each cycle of the free chlorine measurement sequence comprises an applied voltage of approximately 0.40 volt having a duration of approximately 450 milliseconds. A second portion 634 of each cycle of the free chlorine measurement sequence comprises an applied voltage of approximately 0.8 volt having a duration of approximately 200 milliseconds. A third portion 636 of each cycle of the free chlorine measurement sequence comprises an applied voltage of approximately −1.15 volt having a duration of approximately 230 milliseconds.

During the first portion 632 of each cycle 630 of the free chlorine measurement sequence 612, the magnitude of the current (I_{CELL}) flowing through the cell from the working electrode to the counter electrode is measured by the system controller 42 by sensing a voltage across the voltage sensing resistor 122 of FIG. 5. As discussed above, the applied waveform has a voltage of approximately 0.40 volt during the first portion of each cycle of the free chlorine measurement sequence. The magnitude of the current is determined by the electrons released by reducing hypochlorous acid (HOCl) at the working electrode in accordance with Equation (2). The current is proportional to the reduction and is thus proportional to the concentration of chlorine in the water.

The currents measured during each of the 15 cycles of the free chlorine measurement sequence 612 are averaged to determine an average signal value in nanoamperes. The average signal value is converted to parts per million (ppm) of chlorine using previously determined calibration values for the amperometric sensor system. The previously determined calibration values are stored in the amperometric sensor system during a previous calibration step using known concentrations. The calibration step usually is performed when the amperometric sensor system is assembled in a factory; however, the calibration step may also be performed in the field using known concentrations of chlorine.

The measured concentration FCL of free chlorine may be displayed on the system controller 42. The measured concentration may also be transmitted to a monitoring system (e.g., an interface device) as the raw signal (average current value in nanoamperes) and as a concentration in ppm wherein the concentration is defined as free chlorine (FCL).

### Detection of Monochloramine Plus Free Chlorine

As described below, the same electrodes used to measure free chlorine are used to measure the combined concentrations of monochloramine and free chlorine by modifying the applied waveform during a second step of the method.

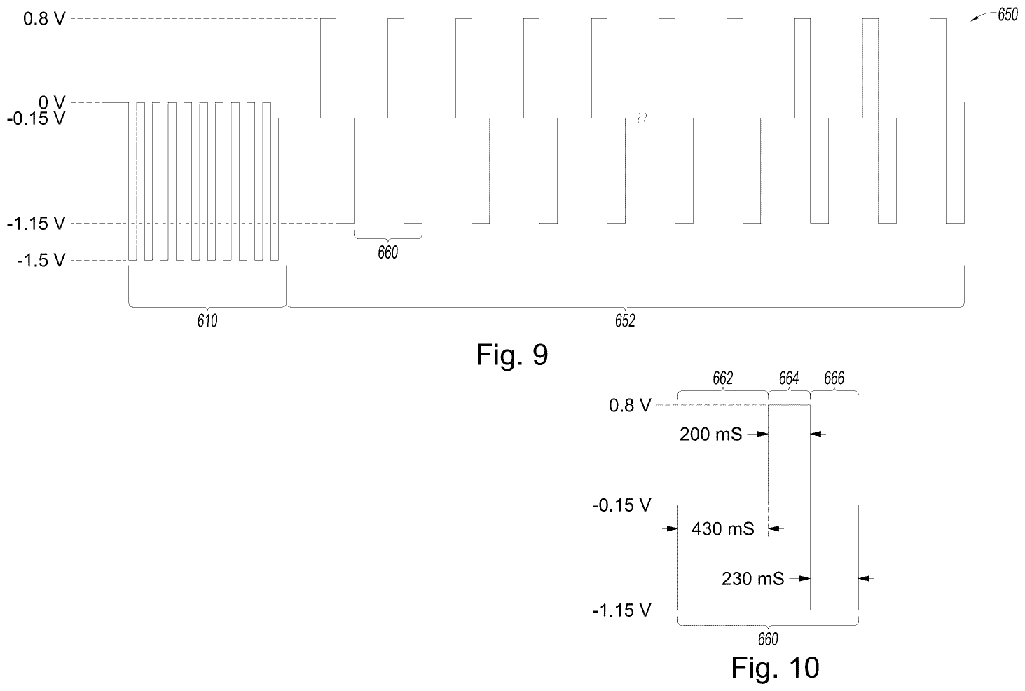

The second step of determining the combined concentrations is illustrated by the applied voltages of a waveform 650 in FIG. 9. The waveform 650 is generated by the waveform generator 210 of FIG. 5 in response to the second mode command from the system controller 42. The waveform comprises the previously described 10-cycle activation sequence 10 followed by a combined concentration measurement sequence 652.

Similar to the free chlorine measurement sequence 612 of FIG. 6, the combined concentration measurement sequence 652 of FIG. 9 comprises a plurality of cycles 660. As discussed above, the number of cycles may vary with different embodiments; however, a sequence of 15 cycles has been found to be sufficient in the illustrated embodiment. Only 10 of the 15 cycles are illustrated in FIG. 9 with a curled break symbol representing the missing 5 cycles. As shown in FIG. 10 for one cycle, each cycle has a first portion 662, a second portion 664, and a third portion 666.

Unlike the first portion 632 of each cycle 630 of the free chlorine measurement sequence, the first portion 662 of each cycle of the combined concentration measurement sequence 650 comprises an applied voltage in a range of approximately −0.15 volt to approximately 0.0 volt having a duration of approximately 450 milliseconds. The second portion of each cycle of the combined concentration measurement sequence comprises an applied voltage of approximately 0.8 volt at a duration of approximately 200 milliseconds. The third portion of each cycle of the combined concentration measurement sequence comprises an applied voltage of approximately −1.15 volt at a duration of approximately 230 milliseconds. In the illustrated embodiment, the second and third portions of each cycle of the combined concentration measurement sequence correspond to the second and third portions of each cycle of the free chlorine measurement sequence.

During the first portion 662 of each cycle 660 of the combined concentration measurement sequence 612, the magnitude of the current (I_{CELL}) flowing through the cell from the working electrode to the counter electrode is measured by sensing a voltage across the voltage sensing resistor 122 of FIG. 5 As discussed above, the applied waveform has a voltage in a range of approximately −0.15 volt to approximately 0.0 volt during the first portion of each cycle of the combined chlorine measurement sequence. The magnitude of the current is determined by the combined electrons released by reducing hypochlorous acid (HOCl) at the working electrode in accordance with Equation (3) and by reducing monochloramine (NH₂Cl) in accordance with Equation (4). The current is proportional to the combined reductions and is thus proportional to the combined concentrations of chlorine and monochloramines in the water. At the selected potential in the range of approximately −0.15 volt to approximately 0.0 volt, dichloramine and trichloramine are not measured.

The currents measured during each of the 15 cycles of the combined concentration measurement sequence 662 are averaged to determine an average signal value in nanoamperes. The average signal value is converted to parts per million (ppm) of the combined concentrations using previously determined calibration values for the amperometric sensor system. The previously determined calibration values are stored in the amperometric sensor system during a previous calibration previous calibration step using known concentrations of chlorine and monochloramine. The calibration step usually is performed when the amperometric sensor system is assembled in a factory; however, the calibration step may also be performed in the field using known concentrations of chlorine and monochloramine.

The measured combined concentration CCL of free chlorine and monochloramine may be displayed on the system controller 42. The measured combined chlorine concentration may also be transmitted to a monitoring system as the raw signal (average current value in nanoamperes) and as a concentration in ppm wherein the concentration is defined as combined chlorine (CCL).

### Determination of Monochloramine

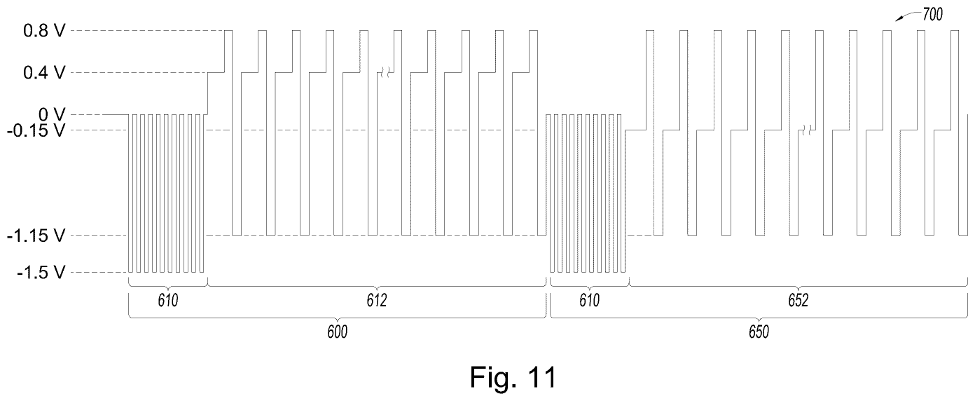

In the illustrated embodiment, the detection of free chlorine using the waveform 612 illustrated in FIG. 6 and the detection of the combined concentrations of free chlorine and monochloramine using the waveform 652 as illustrated in FIG. 9 are performed in succession with the activation sequence 610 performed before the free chlorine measurement sequence 630 and performed again after the free chlorine measurement sequence and before the combined concentration measurement sequence 660. The combined sequences are illustrated by a waveform 700 in FIG. 11.

During the free chlorine measurement sequence 630, the concentration of the free chlorine is determined as the value FCL as described above. During the combined concentration measurement sequence, the combined concentration of free chlorine and monochloramine (total chlorine) is determined as the value CCL as described above. The system controller subtracts the free chlorine value FCL from the total chlorine value CCL to yield a monochloramine fraction (MCL).

The determined concentration (MCL) of monochloramine may be displayed on the system controller 42. The determined concentration may also be transmitted to a monitoring system. In the illustrated embodiment, both the FCL value and the MCL value are displayed on the system controller, on the monitoring system, or on both. In other embodiments, values representing the measured currents may be transmitted to a monitoring system, and the determination of FCL, CCL, and MCL may be performed by the monitoring system.

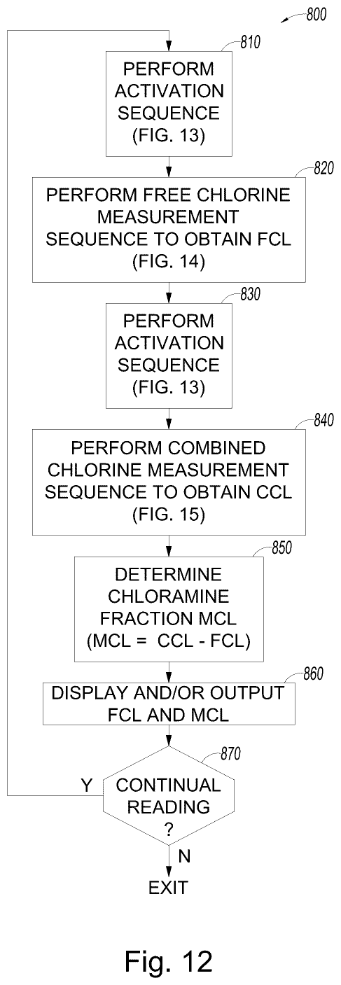

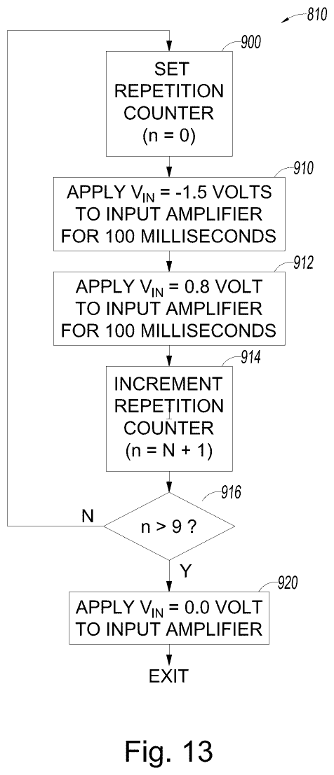

The foregoing concentration measurements and the determination of monochloramine are illustrated by a flowchart 800 in FIG. 12. During a first activation procedure 810, the system controller 42 of FIG. 5 causes the waveform generator 210 to generate the activation sequence 610 illustrated in FIG. 6. The first activation procedure is shown in more detail in FIG. 13.

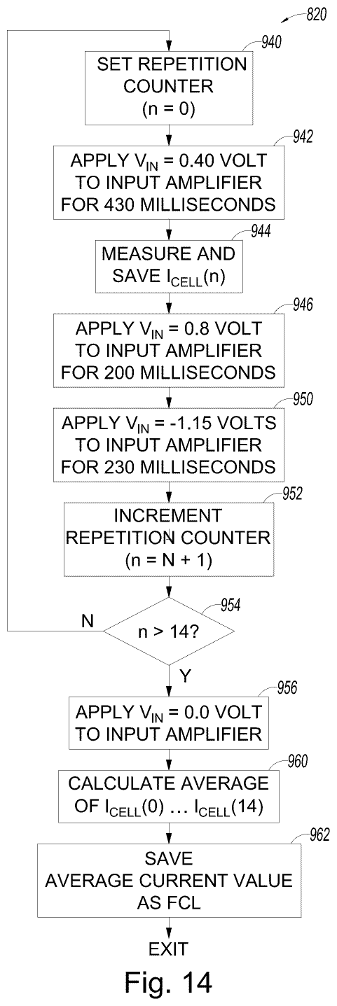

After completing the activation sequence in the first activation procedure 810, the system controller 42 performs a free chlorine measurement procedure 820 during which the system controller causes the waveform generator 210 to generate the waveform 612 of FIG. 6. The system controller measures the current I_{CELL}, which is responsive to the concentration of free chlorine in the water being measured, and determines the concentration FCL of the free chlorine. The free chlorine measurement procedure is shown in more detail in FIG. 14.

After completing the free chlorine measurement procedure 820, the system controller 42 performs a second activation procedure 830, which corresponds to the first activation procedure 810. Accordingly, the second activation procedure is also shown in FIG. 13.

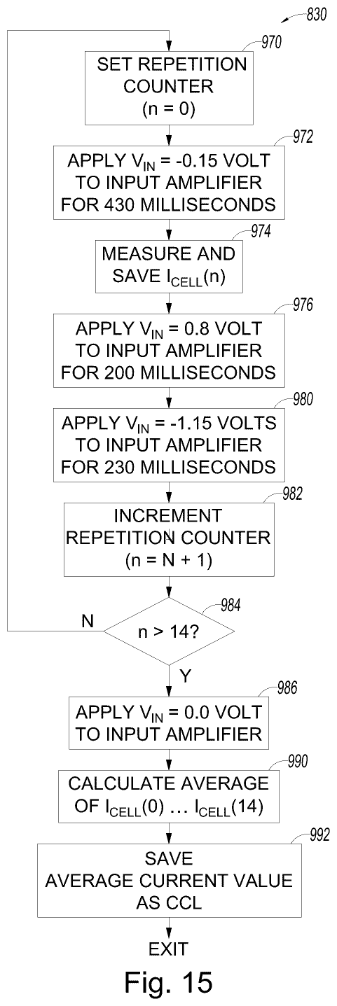

After completing the activation sequence 610 in the second activation procedure 830, the system controller 42 performs a combined (or total) chlorine measurement procedure 840 during which the system controller causes the waveform generator 210 to generate the waveform 652 of FIG. 9. The system controller measures the current I_{CELL}, which is responsive to the combined concentration of free chlorine and monochloramine in the water being measured, and determines the combined concentration CCL of the free chlorine and the chloramine. The combined concentration measurement procedure is shown in more detail in FIG. 15.

After completing the free chlorine measurement procedure 840, the system controller 42 proceeds to a monochloramine determination procedure 850, wherein the system controller determines the monochloramine concentration MCL by subtracting the free chlorine concentration FCL from the combined chlorine concentration CCL (e.g., MCL=CCL−FCL).

After determining the combined chlorine concentration, the system controller 42 proceeds to a display/output procedure 1060 wherein the system controller displays the FCL value and the MCL value on the display 46 or outputs the FCL value and the MCL value or both displays and outputs the two values.

After displaying the FCL value and the MCL value or after outputting the two values or after displaying and outputting the two values, the system controller 42 then determines whether the system controller is operating in a continuous measurement mode or a single measurement mode in a decision block 870. If the system controller is operating in the single measurement mode, only one measurement is required, and the procedure 800 in FIG. 12 exits as shown. However, if the system controller is operating in a continual measurement mode, the procedure in FIG. 12 returns to the first activation procedure 810 and repeats the procedure until the mode is changed or the system controller receives an instruction to halt. In the continuous measurement mode, the system controller updates the displayed FCL and MCL values, outputs the two values or displays and outputs the two values at the conclusion of each pass through the procedure of FIG. 12. In the illustrated embodiment, the total time required to perform the two activation sequences and the two measurement sequences can be as short as 29.5 seconds. Assuming, for example, a processing overhead of approximately 500 milliseconds, the measurements and calculations can be performed every 30 seconds such that the display 46 of FIG. 1 can be updated every 30 seconds if desired. If some are all of the dwell times between changes in waveforms are increased or decreased, the update time is increased or decreased accordingly.

FIG. 13 illustrates the first activation procedure 810 of FIG. 12 in more detail. In a first step 900, the waveform generator 210 sets a repetition counter value n to 0. Then, in a first voltage application step 910, the waveform generator applies −1.50 volts as the voltage V_{IN} to the noninverting input (+) of the input amplifier 150 of FIG. 5 for 100 milliseconds. Then, in a second voltage application step 912, the waveform generator applies 0.8 volt as the voltage V_{IN} to the input amplifier for 100 milliseconds. The waveform generator increments the repetition counter value n by 1 (e.g., n=n+1) in a step 914. In a decision step 916, the waveform generator determines whether the repetition counter value is greater than 9 to determine whether the waveform generator has applied the pairs of voltage more than 10 times. If the repetition counter value is not greater than 9, the waveform generator returns from the decision step to the first voltage activation step 910 to repeat the applications of the two voltages of the activation procedure. This looping continues until the repetition counter value is greater than 9. When the repetition counter value is greater than 9, the waveform generator proceeds from the decision step to a voltage application step 920 wherein the waveform generator applies 0 volt to the input amplifier. The waveform generator then exits the procedure. The flowchart of FIG. 13 also illustrates the second activation procedure 830 of FIG. 12.

FIG. 14 illustrates the free chlorine measurement procedure 820 of FIG. 12. In a first step 940, the system controller sets a repetition counter value n to 0. Then, in a first voltage application step 942, the waveform generator 210 applies 0.40 volt as the voltage V_{IN} to the noninverting input (+) of the input amplifier 150 of FIG. 5 for 450 milliseconds. The applied voltage may be slightly less than 0.40 volt (e.g., approximately 0.35 volt) or slightly more than 0.40 volt (e.g., up to approximately 0.45 volt). While this voltage is applied, the system controller 42 measures the voltage across the sensing resistor 122 of FIG. 5 in a current determining step 944 to determine the current I_{CELL} flowing through the sensing resistor. The determined current I_{CELL}(n) in nanoamperes (nA) is saved. Then, in a second voltage application step 946 the waveform generator applies 0.8 volt as the voltage V_{IN} to the input amplifier for 200 milliseconds. Then, in a third voltage application step 950, the waveform generator applies −1.15 volts as the voltage V_{IN} to the input amplifier for 230 milliseconds. The waveform generator increments the repetition counter value n by 1 (e.g., n=n+1) in a step 952. In a decision step 954, the waveform generator determines whether the repetition counter value is greater than 14 to determine whether the waveform generator has applied the cycle of three voltages 15 times. If the repetition counter value is not greater than 14, the waveform generator returns from the decision step to the first voltage application step 942 to repeat the applications of the three voltages of the free chlorine measurement procedure. The current is measured during each respective first voltage application step. This looping continues until the repetition counter value is greater than 14. When the repetition counter value is greater than 14, the waveform generator proceeds from the decision step to a voltage application step 956 wherein the waveform generator applies 0 volt to the input amplifier. Then, in a step 960, the system controller 42 calculates an average current value from the 15 current values (I_{CELL}(0), I_{CELL}(1) . . . I_{CELL}(14)) saved in the 15 current determining steps 944 described above. In a step 962, the average current value in nanoamperes is saved as the free chlorine concentration value FCL.

FIG. 15 illustrates the combined chlorine measurement procedure 830 of FIG. 12, which begins when the system controller 42 sends the second mode command to the waveform generator 210. In a first step 970, the waveform generator sets a repetition counter value n to 0. Then, in a first voltage application step 972, the waveform generator 210 applies −0.15 volts as the voltage V_{IN} to the noninverting input (+) of the input amplifier 150 of FIG. 5 for 450 milliseconds. As noted above, the voltage can range from approximately −0.15 volt to approximately 0.0 volt. While this voltage is applied, the system controller measures the voltage across the sensing resistor 122 of FIG. 5 in a current determining step 974 to determine the current I_{CELL} flowing through the sensing resistor. The determined current I_{CELL}(n) in nanoamperes is saved. Then, in a second voltage application step 976, the waveform generator applies 0.8 volt as the voltage V_{IN} to the input amplifier for 200 milliseconds. Then, in a third voltage application step 980, the waveform generator applies −1.15 volts as the voltage V_{IN} to the input amplifier for 230 milliseconds. The waveform generator increments the repetition counter value n by 1 (e.g., n=n+1) in a step 982. In a decision step 984, the waveform generator determines whether the repetition counter value is greater than 14 to determine whether the waveform generator has applied the cycle of three voltages 15 times. If the repetition counter value is not greater than 14, the waveform generator returns from the decision step to the first voltage application step 972 to repeat the applications of the three voltages of the combined measurement procedure. The current is measured during each respective first voltage application step. This looping continues until the repetition counter value is greater than 14. When the repetition counter value is greater than 14, the waveform generator proceeds from the decision step to a voltage application step 986 wherein the waveform generator applies 0 volt to the input amplifier. Then, in a step 990, the system controller generates an average current value from the 15 current values (I_{CELL}(0), I_{CELL}(1) . . . I_{CELL}(14)) saved in the 15 current determining steps 974 described above. In a step 992, the average current value in nanoamperes is saved as the combined chlorine concentration value CCL.

After saving the combined chlorine concentration value CCL in the step 992 of FIG. 15, the system controller 42 proceeds to the procedure 840 of FIG. 12 wherein the system controller determines the monochloramine concentration MCL by subtracting the free chlorine concentration FCL from the combined chlorine concentration CCL (e.g., MCL=CCL−FCL). The free chlorine concentration FCL and the monochloramine concentration MCL can be displayed locally on the display 46 of FIG. 1 and may be output via the communications interface 48.

Although described above with respect to repetition counters in the procedures, it should be understood that the various waveforms can be generated by outputting voltage values from an indexed table. It should be further understood that the waveform generator 210 and the system controller 42 may be combined in a single microcontroller or the like. It should be further understood that the combined chlorine measurement may be performed before the free chlorine measurement. It should be further understood that the voltages identified herein represent optimal voltages within ranges of voltages. For example, as described above, the first cycle portion of the free chlorine measurement waveform 812 may vary from the optimal voltage of 0.40 volt to as low as 0.35 volt and to as high as 0.45 volt. In like manner, the first cycle portion of the combined chlorine measurement may vary from the optimal voltage of −0.15 volt to as low as −0.20 volt to as high as +0.05 volt.

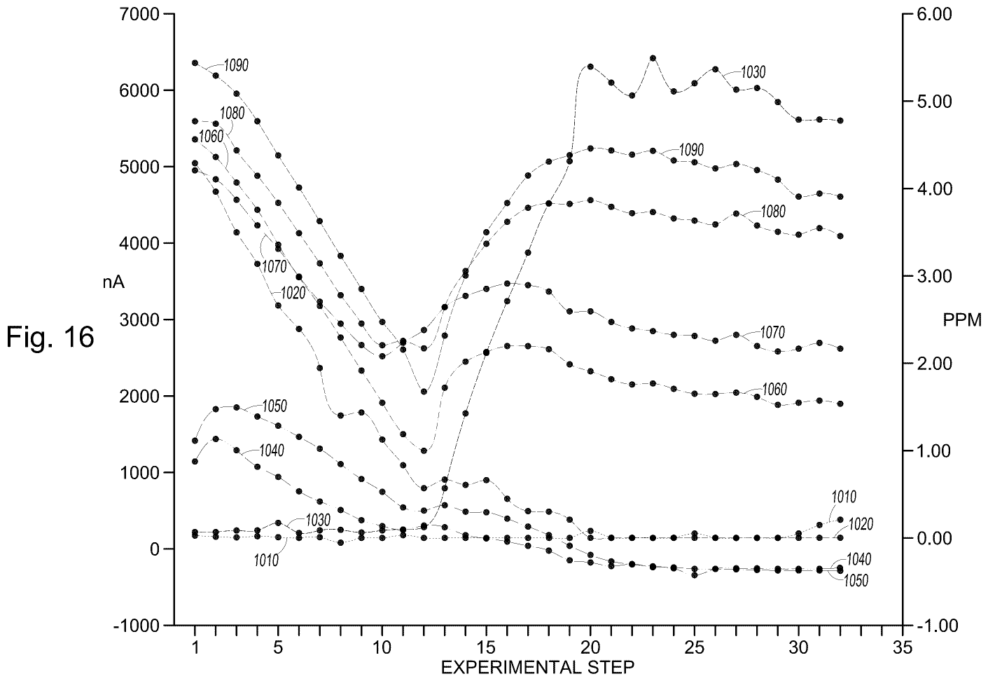

FIG. 16 illustrates graphs of the operation of the above-described apparatus and method in comparison to measurements made using a commercially available tester. The illustrated comparison graphs were made using an SL1000 Portable Parallel Analyzer (PPA) Portable Colorimeter from Hach Company of Loveland, Colorado. The horizontal axis in FIG. 16 represents experimental steps from a step 1 to a step 32. The right vertical axis represents the measured concentrations in part-per-million (PPM) as measured by the commercial tester. The left vertical axis represents the current in nanoamperes (nA) as measured by the apparatus and method disclosed herein. The graphs result from injecting water having a substantially constant 5 PPM concentration of sodium hypochlorite into a sample chamber (not shown). During each step, the concentrations of free chlorine and monochloramine within the chamber is measured using the commercial tester; and the concentrations of free chlorine and combined chlorine are measured using the apparatus and method disclosed herein. At each step, an additional concentration of ammonia (NH₃) (e.g., approximately 0.24 PPM) is added to the chamber. The added ammonia rapidly reacts with the free chlorine to form monochloramine or dichloramine. Thus, the concentration of free ammonia remains near 0 as represented by a first graph 1010 until all free chlorine has reacted.

As the concentration of ammonia increases in each step, the concentration of free chlorine measured by the commercial tester decreases as represented by a second graph 1020 having magnitudes in PPM as shown by the right vertical axis. The concentration of free chlorine continues to decrease with increasing concentrations of ammonia as shown in steps 2 through 20 at which concentration, substantially all of the free chlorine is converted to monochloramine.

The concentration of monochloramine as measured by the commercial tester is represented by a third graph 1030, which also has magnitudes in PPM as shown by the right vertical axis. As illustrated in FIG. 16, very little monochloramine is produced in steps 1 through 12 After step 12, the concentration of monochloramine increases until around step 18. After step 18, the concentration of monochloramine remains somewhat constant around 5 PPM because all of the free chlorine in the input stream reacts with the concentration of ammonia at that step. The introduction of additional ammonia does not produce additional reaction The absence of monochloramine in steps 1 through 12 may be attributed to the generation of dichloramines, which are not measured by the commercial tester.

The measurements obtained by the apparatus and method disclosed herein are represented by a fourth graph 1040, a fifth graph 1050, a sixth graph 1060, a seventh graph 1070, an eighth graph 1080, and a ninth graph 1090. The magnitudes shown in the third through ninth graphs are in nanoamperes (nA) as shown by the left vertical axis.

The fourth graph 1040 represents the averaged currents measured during the first portion 632 of each cycle of the free chlorine measurement sequence 600 as shown in FIG. 8 with the magnitude of the voltage during the first portion selected to be approximately 0.45 volt. The fifth graph 1050 represents the averaged currents measured during the first portion 632 of each cycle of the free chlorine measurement sequence with the magnitude of the voltage during the first portion selected to be approximately 0.40 volt. As illustrated by the two graphs, the measurement at the lower voltage as represented by the fifth graph has better resolution than the measurement at the higher voltage as represented by the fourth graph.

The sixth graph 1060 represents the averaged currents measured during the first portion 662 of each cycle of the combined concentration measurement sequence 650 as shown in FIG. 10 with the magnitude of the voltage during the first portion selected to be approximately 0.0 volt. The seventh graph 1070 represents the averaged currents measured during the first portion 662 of each cycle of the combined concentration measurement sequence 650 as shown in FIG. 10 with the magnitude of the voltage during the first portion selected to be approximately −0.05 volt. The eighth graph 1080 represents the averaged currents measured during the first portion 662 of each cycle of the combined concentration measurement sequence 650 as shown in FIG. 10 with the magnitude of the voltage during the first portion selected to be approximately −0.10 volt. The ninth graph 1090 represents the averaged currents measured during the first portion 662 of each cycle of the combined concentration measurement sequence 650 as shown in FIG. 10 with the magnitude of the voltage during the first portion selected to be approximately −0.15 volt. As illustrated, the four graphs of the combined chlorine measurement have shapes similar to the shapes of the graph of the monochloramine measurement by the commercial tester. The ninth graph 1090 representing measurements at approximately −0.15 volt more closely resembles the shape of monochloramine measurement and is selected as the preferred measurement voltage for the apparatus and method disclosed herein. As discussed above, the measured currents in nanoamperes are converted to concentrations in PPM and the free chlorine concentrations are subtracted from the combined chlorine concentrations to obtain the monochloramine concentrations.

The previous detailed description has been provided for the purposes of illustration and description. Thus, although there have been described particular embodiments of a new and useful invention, it is not intended that such references be construed as limitations upon the scope of this invention except as set forth in the following claims.

We claim:

1. A method for determining a concentration of free chlorine in water using a three-electrode amperometric sensor with a bare working electrode, the amperometric sensor including a potentiostat with selectable voltages, the method comprising:
executing an activation procedure comprising applying an activation voltage sequence to the working electrode, the activation voltage sequence comprising a plurality of activation cycles, each activation cycle comprising a first activation voltage followed by a second activation voltage;
applying a free chlorine measurement sequence to the working electrode, the free chlorine measurement sequence comprising a plurality of free chlorine measurement cycles, each free chlorine measurement cycle comprising a first free chlorine measurement voltage followed by a second free chlorine measurement voltage followed by a third free chlorine measurement voltage;
while the first free chlorine measurement voltage is applied in each free chlorine measurement cycle, measuring a sensed current and saving a respective free chlorine measurement value responsive to the sensed current for each cycle;
averaging the saved free chlorine measurement values to obtain an average free chlorine measurement value; and
converting the average free chlorine measurement value to a value representing a concentration of free chlorine.

2. A method for determining a concentration of monochloramine in water using a three-electrode amperometric sensor with a bare working electrode, the amperometric sensor including a potentiostat with selectable voltages, the method comprising:
executing an activation procedure comprising applying an activation voltage sequence to the working electrode, the activation voltage sequence comprising a plurality of activation cycles, each activation cycle comprising a first activation voltage followed by a second activation voltage;
measuring free chlorine concentration by:
applying a free chlorine measurement sequence to the working electrode, the free chlorine measurement sequence comprising a plurality of free chlorine measurement cycles, each free chlorine measurement cycle comprising a first free chlorine measurement voltage followed by a second free chlorine measurement voltage followed by a third free chlorine measurement voltage;
while the first free chlorine measurement voltage is applied in each free chlorine measurement cycle, measuring a sensed current and saving a respective free chlorine measurement value responsive to the sensed current for each cycle;
averaging the saved free chlorine measurement values to obtain an average free chlorine measurement value; and
converting the average free chlorine measurement value to a value representing a concentration of free chlorine;
repeating the activation procedure;
measuring combined chlorine concentration by:
applying a combined chlorine measurement sequence to the working electrode, the combined chlorine measurement sequence comprising a plurality of combined chlorine measurement cycles, each combined chlorine measurement cycle comprising a first combined chlorine measurement voltage followed by a second combined chlorine measurement voltage followed by a third combined chlorine measurement voltage;
while the first combined chlorine measurement voltage is applied in each combined chlorine measurement cycle, measuring a sensed current and saving a respective combined chlorine measurement value responsive to the sensed current for each cycle;
averaging the saved combined chlorine measurement values to obtain an average combined chlorine measurement value; and
converting the average combined chlorine measurement value to a value representing a concentration of combined chlorine; and
subtracting the value representing the concentration of free chlorine from the value representing the concentration of combined chlorine to determine a concentration of monochloramine.

3. A method for determining a concentration of monochloramine in water using a three-electrode amperometric sensor with a bare working electrode, the amperometric sensor including a potentiostat with selectable voltages, the method comprising:
executing an activation procedure wherein the activation procedure comprises:
applying a first activation voltage to the working electrode for a first activation step time duration;
applying a second activation voltage to the working electrode for a second activation step time duration; and
repeatedly applying the first activation voltage and the second activation voltage for a first plurality of times;
measuring free chlorine concentration wherein measuring free chlorine concentration comprises:
applying a first free chlorine measurement voltage to the working electrode for a first free chlorine measurement duration;
while the first free chlorine measurement voltage is applied, measuring a sensed current and saving a value responsive to the sensed current;
applying a second free chlorine measurement voltage to the working electrode for a second free chlorine measurement duration;
applying a third free chlorine measurement voltage to the working electrode for a third free chlorine measurement duration;
repeatedly applying the first chlorine measurement voltage, measuring the sensed current, applying the second chlorine measurement voltage, and applying the third chlorine measurement voltage for a second plurality of times;
averaging the saved values responsive to the sensed current to obtain a first average sensed current value; and
converting the first average sensed current value to a value representing a concentration of free chlorine;
repeating the activation procedure;
measuring combined chlorine concentration wherein measuring combined chlorine concentration comprises:
applying a first combined chlorine measurement voltage to the working electrode for a first combined chlorine measurement duration;
while the first combined chlorine measurement voltage is applied, measuring a sensed current and saving a value responsive to the sensed current;
applying a second combined chlorine measurement voltage to the working electrode for a second combined chlorine measurement duration;
applying a third combined chlorine measurement voltage to the working electrode for a first combined chlorine measurement duration;
repeatedly applying the first combined chlorine measurement voltage, measuring the sensed current, applying the combined second chlorine measurement voltage, and applying the third combined chlorine measurement voltage for a third plurality of times;
averaging the saved values responsive to the sensed current to obtain a second average sensed current value; and
converting the second average sensed current value to a value representing a combined concentration of free chlorine and monochloramine;
and
subtracting the value representing the concentration of free chlorine from the value representing the combined concentration of free chlorine and monochloramine to determine a concentration of monochloramine.

4. The method as defined in claim 3 wherein:
the first activation voltage is approximately −1.5 volts; and
the second activation voltage is approximately 0.0 volt.

5. The method as defined in claim 4 wherein:
the first activation step time duration is approximately 100 milliseconds; and
the second activation step time duration is approximately 100 milliseconds.

6. The method as defined in claim 3 wherein the first plurality of times is 10.

7. The method as defined in claim 3 wherein:
the first free chlorine measurement voltage is in a range of approximately 0.35 volt to approximately 0.45 volt;
the second free chlorine measurement voltage is approximately 0.8 volt; and
the third free chlorine measurement voltage is approximately −1.15 volts.

8. The method as defined in claim 7 wherein:
the first free chlorine measurement duration is approximately 430 milliseconds;
the second free chlorine measurement duration is approximately 200 milliseconds; and
the third free chlorine measurement duration is approximately 230 milliseconds.

9. The method as defined in claim 3 wherein the second plurality of times is 15.

10. The method as defined in claim 3 wherein:
the first combined chlorine measurement voltage is in a range of approximately −0.15 volt to approximately 0.0 volt;
the second combined chlorine measurement voltage is approximately 0.8 volt; and
the third combined chlorine measurement voltage is approximately −1.15 volts.

11. The method as defined in claim 10 wherein:
the first combined chlorine measurement duration is approximately 430 milliseconds;
the second combined chlorine measurement duration is approximately 200 milliseconds; and
the third combined chlorine measurement duration is approximately 230 milliseconds.

12. The method as defined in claim 3 wherein the third plurality of times is 15.

13. A method for determining a concentration of monochloramine in water using a three-electrode amperometric sensor with a bare working electrode, the amperometric sensor including a potentiostat with selectable voltages, the method comprising:
executing an activation procedure wherein the activation procedure comprises 10 activation cycles, each activation cycle comprising:
applying −1.5 volts to the working electrode for 100 milliseconds; and
applying a voltage of approximately 0.0 volt to the working electrode for 100 milliseconds;
executing a free chlorine concentration measurement procedure comprising:
executing 15 free chlorine measurement cycles, each free chlorine measurement cycle comprising:
applying a voltage in a range of approximately 0.35 volt to approximately 0.45 volt to the working electrode for 430 milliseconds as a free chlorine measurement voltage;
while the free chlorine measurement voltage is applied, measuring a sensed current and saving a value responsive to the sensed current;
applying 0.8 volt to the working electrode for 200 milliseconds;
applying −1.15 volts to the working electrode for 230 milliseconds;
averaging the saved values responsive to the sensed current to obtain a first average sensed current value; and
converting the first average sensed current value to a value representing a concentration of free chlorine;
repeating the activation procedure;
executing a combined chlorine concentration measurement procedure comprising:
executing 15 combined chlorine measurement cycles, each combined chlorine measurement cycle comprising:
applying a voltage in a range of approximately −0.15 volt to approximately 0.0 volt to the working electrode for 430 milliseconds as a combined chlorine measurement voltage;
while the combined chlorine measurement voltage is applied, measuring a sensed current and saving a value responsive to the sensed current;
applying 0.8 volt to the working electrode for 200 milliseconds;
applying −1.15 volts to the working electrode for 230 milliseconds;
averaging the saved values responsive to the sensed current to obtain a second average sensed current value; and
converting the second average sensed current value to a value representing a concentration of combined chlorine;
and
subtracting the value representing the concentration of free chlorine from the value representing the concentration of combined chlorine to determine a concentration of monochloramine.

14. An amperometric system for determining concentrations of free chlorine and monochloramine in water comprising:
a three-electrode potentiostat comprising:
a measurement cell having a working electrode, a reference electrode and a counter electrode, the electrodes insertable into a source of water;
circuitry responsive to an input voltage to generate a current through the measurement cell to cause a voltage between the working electrode and the reference electrode responsive to the input voltage; and
a current sensor that senses the current through the measurement cell;
a waveform generator having an output coupled to the potentiostat, the waveform generator generating the input voltage as a first waveform, a second waveform, a third waveform, and a fourth waveform applied in sequence, wherein:
the first waveform comprises 10 cycles, each cycle comprising a first half cycle at −1.5 volts for 100 milliseconds and a second half cycle of 0.0 volt at 100 milliseconds;
the second waveform comprises 15 cycles, each cycle comprising a first cycle portion, a second cycle portion and a third cycle portion, the first cycle portion comprising a voltage in a range of approximately 0.35 volt to approximately 0.45 volt applied for 430 milliseconds, the second cycle portion comprising 0.8 volt applied for 200 milliseconds, the third cycle portion comprising −1.15 volts applied for 230 milliseconds;
the third waveform comprises 10 cycles, each cycle comprising a first half cycle at −1.5 volts for 100 milliseconds and a second half cycle of 0.0 volt at 100 milliseconds;
the fourth waveform comprises 15 cycles, each cycle comprising a first cycle portion, a second cycle portion and a third cycle portion, the first cycle portion comprising a voltage in a range of approximately −0.15 volt to approximately 0.0 volt applied for 430 milliseconds, the second cycle portion comprising 0.8 volt applied for 200 milliseconds, the third cycle portion comprising −1.15 volts applied for 230 milliseconds;
and
a system controller coupled to the potentiostat to receive values from the current sensor, the system controller configured to:
receive and save a respective value responsive to a concentration of free chlorine during each first cycle portion of the second waveform;
averaging the saved values determine a first average sensed current value;
convert the first average sensed current value to a value representing the concentration of free chlorine;
receive and save a respective value responsive to a concentration of total chlorine during each first cycle portion of the fourth waveform;
averaging the saved values determine a second average sensed current value;
convert the second average sensed current value to a value representing a concentration of combined chlorine and monochloramine; and
subtract the value representing the concentration of free chlorine from the value representing the concentration of combined chlorine and monochloramine to determine a concentration of monochloramine.

15. A method for determining a first concentration of a first parameter and determining a second concentration of a second parameter in water using a three-electrode amperometric sensor with a bare working electrode, the amperometric sensor including a potentiostat with selectable voltages, the method comprising:
executing an activation procedure comprising applying an activation voltage sequence to the working electrode, the activation voltage sequence comprising a plurality of activation cycles, each activation cycle comprising a first activation voltage followed by a second activation voltage;
measuring the first concentration of the first parameter by:
applying a first parameter measurement sequence to the working electrode, the first parameter measurement sequence comprising a plurality of first parameter measurement cycles, each first parameter measurement cycle comprising an initial first parameter measurement voltage followed by a second first parameter measurement voltage followed by a third first parameter measurement voltage;
while the initial first parameter measurement voltage is applied in each first parameter measurement cycle, measuring a sensed current and saving a respective first parameter measurement value responsive to the sensed current for each cycle;
averaging the saved first parameter measurement values to obtain an average first parameter measurement value; and
converting the average first parameter measurement value to a value representing a first concentration of the first parameter;
repeating the activation procedure;
measuring a combined concentration of the first parameter and the second parameter by:
applying a combined concentration measurement sequence to the working electrode, the combined concentration measurement sequence comprising a plurality of combined concentration measurement cycles, each combined concentration measurement cycle comprising a first combined concentration measurement voltage followed by a second combined concentration measurement voltage followed by a third combined concentration measurement voltage;
while the first combined chlorine measurement voltage is applied in each combined concentration measurement cycle, measuring a sensed current and saving a respective combined concentration measurement value responsive to the sensed current for each cycle;
averaging the saved combined concentration measurement values to obtain an average combined concentration measurement value; and
converting the average combined concentration measurement value to a value representing a combined concentration of the first parameter and the second parameter;
and
subtracting the value representing the concentration of the first parameter from the value representing the combined concentration of the first parameter and the second parameter to determine a concentration of the second parameter.

16. The method as defined in claim 15 wherein the first parameter is free chlorine and the second parameter is monochloramine.
